(19) United States
(12) Patent Application Publication
(10) Pub. No.: US 2024/0277356 A1
(43) Pub. Date: Aug. 22, 2024

(54) CYCLIC ASPIRATION SYSTEM PRODUCING A CYCLIC ASPIRATION PRESSURE WAVEFORM USING A DUAL PRESSURE GENERATOR RECEPTACLE IN LIEU OF A VACUUM PUMP

(71) Applicant: Neuravi Limited, Galway (IE)

(72) Inventors: David VALE, Barna (IE); Tommy GIBBONS, Galway (IE); Alan CARNEY, Galway (IE)

(73) Assignee: Neuravi Limited, Galway (IE)

(21) Appl. No.: 18/441,525

(22) Filed: Feb. 14, 2024

Related U.S. Application Data
(60) Provisional application No. 63/447,506, filed on Feb. 22, 2023.

(51) Int. Cl. A61B 17/22; A61B 17/00

(52) U.S. Cl. CPC A61B 17/22; A61B 2017/0023; A61B 2017/00398; A61B 2017/00544; A61B 2017/00561; A61B 2017/22079; A61B 2217/005

(57) ABSTRACT

Cyclic aspiration system producing a cyclic aspiration pressure waveform of intermittent cycles of vacuum pressure below atmospheric pressure and positive pressure higher than vacuum pressure. The system includes a dual pressure generator receptacle disposed proximally of and connected in fluid communication with an aspiration catheter. The dual pressure generator receptacle receiving a collectable fluid therein that is intermittently cyclically subjectable to application or withdraw of a compressible force via a linear displacement mechanism. Positive pressure is generated while the collectable fluid in the dual pressure generator receptacle is subject to the compressive force and vacuum pressure is generated when not subject to the compressive force. The cyclic aspiration pressure waveform is produced via the dual pressure generator receptacle without a separate vacuum pump.

### CROSS-REFERENCE TO RELATED APPLICATION

This application claims the benefit of U.S. Provisional Patent Application No. 63/447,506, filed on Feb. 22, 2023, the disclosure of which is incorporated herein by reference in its entirety.

### FIELD

The present disclosure generally relates to a system and method used during thrombectomy procedures for the capture and removal of occlusions or clots. Specifically, the present disclosure relates to a cyclic aspiration system for the capture and removal of occlusions or clots in a vessel where the cyclic aspiration pressure waveform includes intermittent cyclic intervals of vacuum pressure (i.e., below atmospheric pressure) and positive pressure (i.e., higher than vacuum pressure, possibly higher than atmospheric pressure). The cyclic aspiration system produces the cyclic aspiration waveform without use of a vacuum pump (e.g., centrifugal pump, piston pump, or diaphragm pump). In particular, the present disclosure is directed to a cyclic aspiration system and method in which the cyclic aspiration pressure waveform is generated using a dual pressure generator receptacle associated with inlet tubing or a rotating hemostatic valve (RHV) disposed proximally of the aspiration catheter, wherein fluid collectable in the dual pressure generator receptacle is intermittently cyclically subjectable to application or withdraw of a compressible force via a linear displacement mechanism to produce both the vacuum pressure interval and the positive pressure interval of the cyclic aspiration pressure waveform, without the use of a vacuum pump (e.g., centrifugal pump, piston pump, or diaphragm pump).

### BACKGROUND

Pulsatile or cyclic aspiration applies a cyclic pressure waveform of intermittent cyclic minimum/low/vacuum/aspiration pressure and maximum/peak/high pressure. During cycles under the minimum/low/vacuum/aspiration pressure the clot is drawn in the proximal direction and captured at the distal tip/end of the aspiration catheter, whereas during cycles of maximum/peak/high pressure the clot is pushed in the distal direction. When utilizing pulsatile or cyclic aspiration during the capture and removal of the clot it is desirable to maximize the cycling frequency of the cyclic pressure waveform and thus maximize clot vibration thereby optimizing aspiration performance. One key challenge in maximizing the cycling frequency is a particular response time required for mechanical actuation of each active component limiting an extent to which the cycling frequency may be increased. Complex conventional systems for maximizing cycling frequency have many active components each required to await their response times before being activated to maintain normal operation. Accordingly, in complex systems with many active components the extent to which the cycling frequency may be maximized is undesirably curtailed. Another concern is that conventional aspiration systems are prone to clogging by the captured clot.

It is therefore desirable to develop an improved cyclic aspiration system utilizing as few active components as possible with an associated maximized response time to attain maximum cycling frequency while also minimizing dampening or decay of the positive pressure wave as well as the additional benefit of reducing the overall cost of manufacture. Still further desirable is to develop an improved cyclic aspiration system preventing, or minimizing, risk of clogging.

### SUMMARY

An aspect of the present disclosure relates to a pulsatile or cyclic aspiration system producing a cyclic aspiration pressure waveform of intermittent cyclic intervals of vacuum pressure below atmospheric pressure and positive pressure higher than vacuum pressure (higher than vacuum pressure, possibly higher than atmospheric pressure) using as few active components as possible with an associated maximized response time to attain maximum cycling frequency while also minimizing dampening or decay of the positive pressure wave as well as the additional benefit of reducing the overall cost of manufacture.

Another aspect of the present disclosure is directed to a cyclic aspiration system for producing a cyclic aspiration pressure waveform without using a vacuum pump (e.g., centrifugal pump, piston pump, or diaphragm pump).

Still another aspect of the present disclosure relates to an improved cyclic aspiration system for producing a cyclic aspiration pressure waveform using a dual pressure generator receptacle as a single device to generate intervals of both vacuum pressure below atmospheric pressure as well as positive pressure higher than vacuum pressure of the cyclic aspiration pressure waveform, without using a vacuum pump (e.g., centrifugal pump, piston pump, or diaphragm pump).

Yet another aspect of the present disclosure is directed to an improved cyclic aspiration system for producing a cyclic aspiration pressure waveform wherein fluid (e.g., blood and/or saline) collectable in the dual pressure generator receptacle is subject to application or withdraw of a compressive force.

While another aspect of the present disclosure relates to an improved cyclic aspiration system for producing a cyclic aspiration pressure waveform wherein fluid (e.g., blood and/or saline) collectable in the dual pressure generator receptacle is associated with inlet tubing arranged proximally of the proximal hub of the aspiration catheter or within a vacuum inlet port of a rotating hemostatic valve.

Another aspect of the present disclosure is directed to an improved cyclic aspiration system for producing a cyclic aspiration pressure waveform in which those components contaminated by blood (e.g., collection vessel, syringe/reservoir, plunger or piston, inlet tubing, connector, one-way valve, catheter hub/rotating hemostatic valve (RHV), and aspiration catheter) may be either separately or as an assembled unit/module discarded after a single use; while a linear displacement mechanism is not contaminated by blood and hence reusable.

### BRIEF DESCRIPTION OF THE DRAWINGS

The above and further aspects of the present disclosure are further discussed with reference to the following description in conjunction with the accompanying drawings, in which like numerals indicate like structural elements and features in various figures. The drawings are not necessarily to scale, emphasis instead being placed upon illustrating principles of the present disclosure. The figures depict one or more implementations of the devices, by way of example only, not by way of limitation.

FIG. 1A is an example of a cyclic aspiration system in accordance with the present disclosure generating the cyclic aspiration pressure waveform wherein the dual pressure generator receptable is a syringe connected in fluid communication with inlet tubing proximally of the proximal hub off the aspiration catheter with the collectable fluid in the syringe being internally compressible via a plunger or piston slidable within a barrel of the syringe;

FIG. 1B depicts the example cyclic aspiration system of FIG. 1A during pre-treatment while being prepped by flushing with saline prior to use in capturing the targeted clot;

FIG. 1C depicts the example cyclic aspiration system of FIG. 1A during the generation of the vacuum pressure interval of the cyclic aspiration pressure waveform by retracting the plunger or piston within the barrel of the syringe capturing the clot at the distal tip/end of the aspiration catheter;

FIG. 1D depicts the example cyclic aspiration system of FIG. 1A during the generation of the positive pressure interval of the cyclic aspiration pressure waveform by advancing the plunger or piston within the barrel of the syringe while the clot is lodged at the distal tip/end of the aspiration catheter;

FIG. 1E depicts the example cyclic aspiration system of FIG. 1A including a spring-loaded reciprocating mechanism to intermittently cyclically displace (i.e., retract or advance) the plunger or piston within the barrel of the syringe producing the cyclic aspiration pressure waveform;

FIG. 2A is a side view of another example cyclic aspiration system in accordance with the present disclosure generating the cyclic aspiration pressure waveform wherein the dual pressure generator receptacle is a vacuum inlet port of a rheostatic hemostatic valve connected to the aspiration catheter wherein the volume within the vacuum inlet port is internally compressible via a reciprocating plunger or piston slidable therein; depicted during the generation of the vacuum pressure interval of the cyclic aspiration pressure waveform with the plunger or piston in a retracted state;

FIG. 2B is a side view of the example cyclic aspiration system of FIG. 2A depicted during the generation of the positive pressure interval of the cyclic aspiration pressure waveform with the plunger or piston in an advanced state;

FIG. 3A is a side view of yet another example cyclic aspiration system in accordance with the present disclosure generating the cyclic aspiration pressure waveform wherein the dual pressure generator receptacle is a concertinaed container transitionable from an axially expanded state to an axially collapsed state when subject to an external axial force; depicted during the generation of the vacuum pressure interval of the cyclic aspiration pressure waveform with the concertinaed container in a default axially non-collapsed state (i.e., free from the external axial force);

FIG. 3B is a side view of the example cyclic aspiration system of FIG. 3A during the generation of the positive pressure interval of the cyclic aspiration pressure waveform with the concertinaed container depicted in the axially collapsed state (i.e., subject to the external axial force);

FIG. 4A is still another example cyclic aspiration system in accordance with the present disclosure generating the cyclic aspiration pressure waveform wherein the dual pressure generator receptacle is a liquid reservoir with a collected fluid (e.g., blood and/or saline) therein compressible by a plunger or piston slideable therein; depicted during the vacuum pressure interval of the cyclic aspiration pressure waveform with the plunger or piston in a retracted state within the liquid reservoir; and

FIG. 4B depicts the example cyclic aspiration system of FIG. 4A during the generation of the positive pressure interval of the cyclic aspiration pressure waveform with the plunger or piston in an advanced state within the liquid reservoir.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 71% to 99%.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

The cyclic aspiration system in accordance with the present disclosure produces a cyclic aspiration pressure waveform of intermittent cyclic intervals of vacuum pressure (i.e., pressure below atmospheric pressure) and positive pressure (i.e., pressure higher than vacuum pressure) without using a vacuum pump (e.g., centrifugal pump, piston pump, or diaphragm pump). Instead, the cyclic aspiration system in accordance with the present disclosure produces the cyclic aspiration waveform using a dual pressure generator receptacle associated either with inlet tubing or a rotating hemostatic valve proximally of the aspiration catheter. Collected fluid (e.g., blood and/or saline) within the dual pressure generator receptacle is intermittently cyclically subject to application or withdraw of a compressive force to produce the intervals of both vacuum pressure and positive pressure of the cyclic aspiration pressure waveform.

Several non-limiting examples of the dual pressure generator receptable producing the cyclic aspiration pressure waveform in the cyclic aspiration system without using a vacuum pump (e.g., centrifugal pump, piston pump, or diaphragm pump) in accordance with the present disclosure are illustrated and described herein.

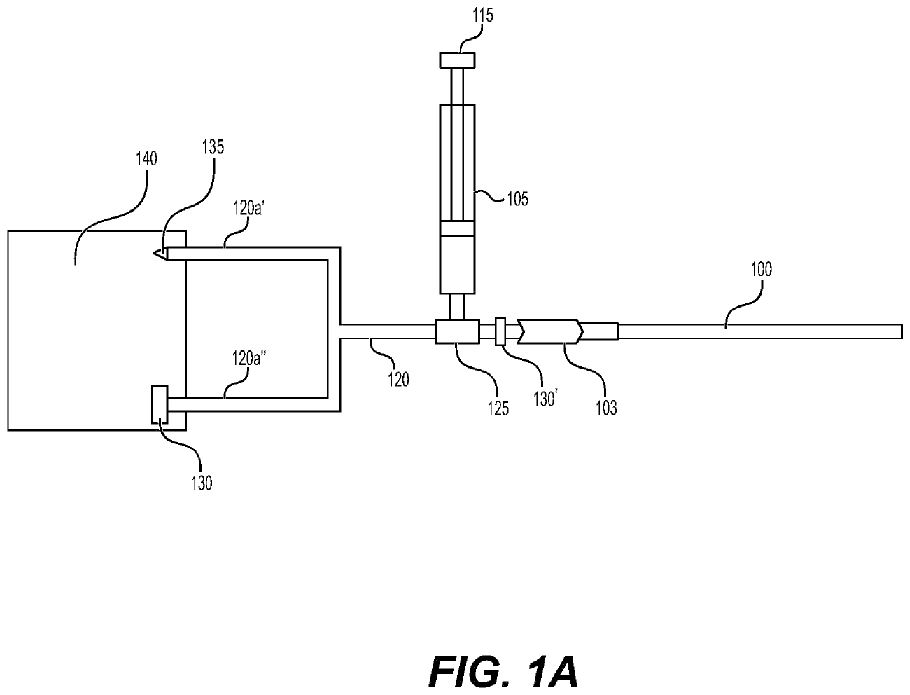

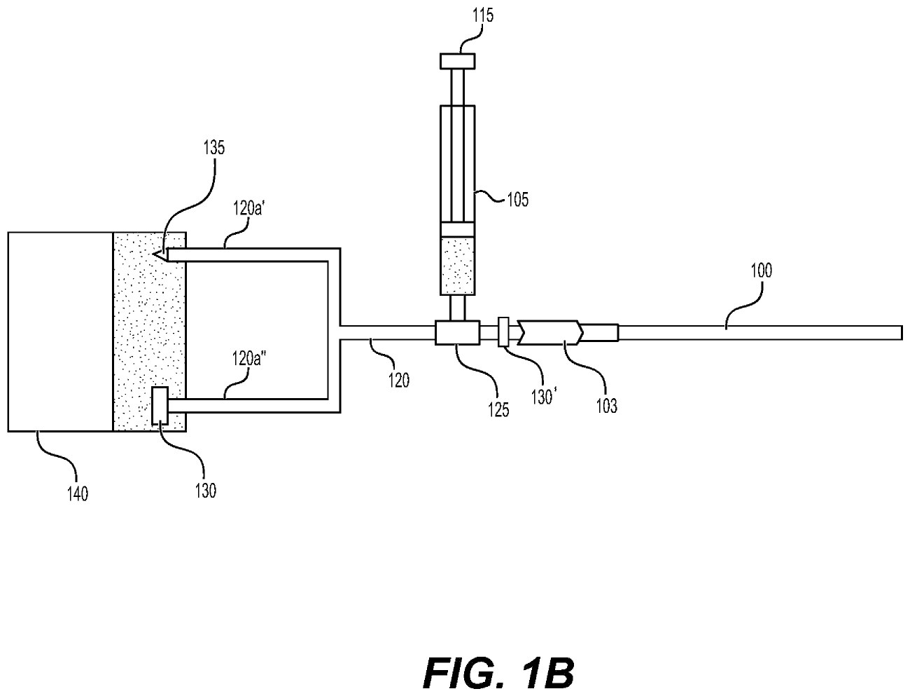

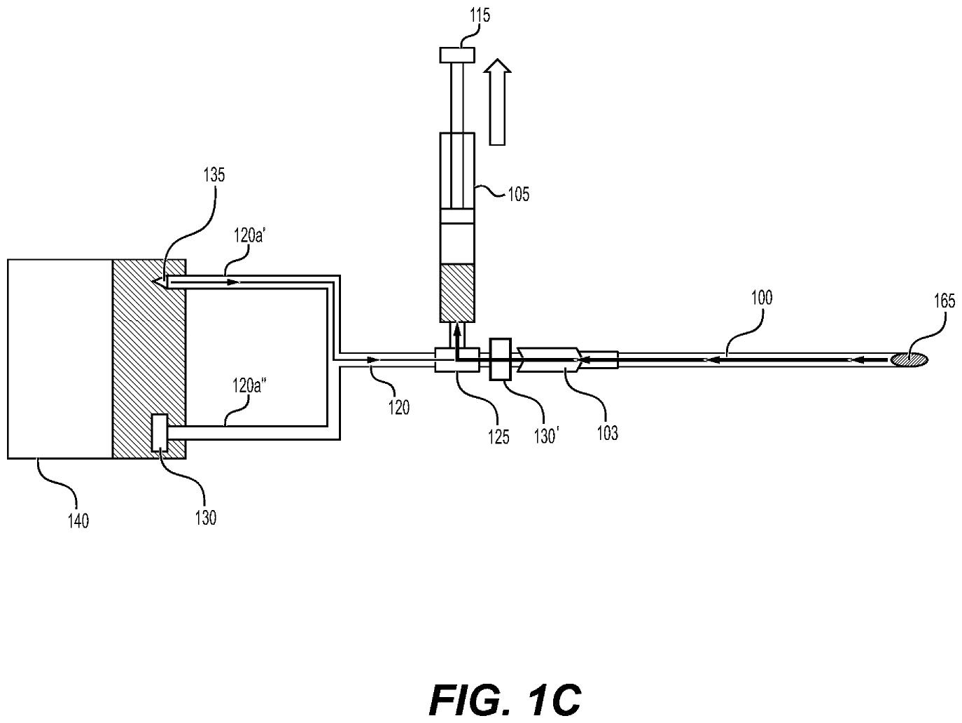

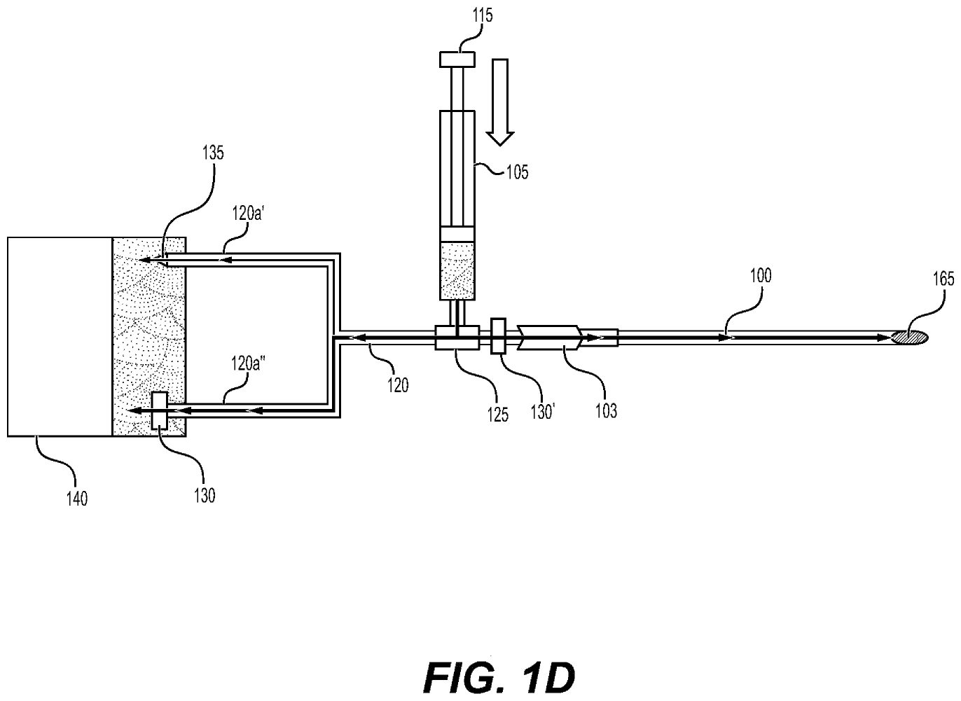

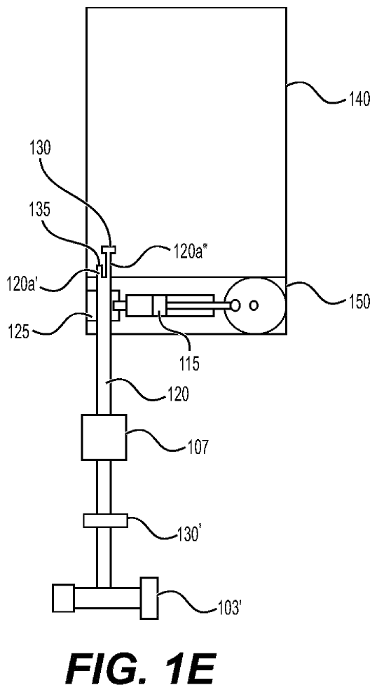

FIG. 1A depicts an example cyclic aspiration system without using a vacuum pump in accordance with the present disclosure wherein the dual pressure generator receptacle is a syringe associated with inlet tubing disposed proximally of a proximal hub attached to the aspiration catheter. A syringe 105 having a displaceable plunger or piston 115 slidable within the barrel of the syringe is connected via a 3-way (e.g., T-shape) connector 125 in fluid communication with inlet tubing 120 and a proximal hub 103 attached to (or an integral part of) the aspiration catheter 100. A one-way valve 130′ is disposed proximal of the proximal hub 103. Retraction of the plunger 115 in the syringe generates vacuum pressure opening the one-way valve 130′ allowing the fluid from the aspiration catheter 100 to be drawn or sucked therethrough. When the plunger 115 is advanced in the syringe 105 the positive pressure pushing towards both the collection vessel 140 and the aspiration catheter 100 closes the one-way valve 130′ preventing fluid from being injected into the aspiration catheter and preventing the clot from being pushed too far forward,

A proximal end of the inlet tubing 120 in the example illustrated is Y-split (depicted in an exaggerated manner in FIG. 1A) into two respective inlet ports including a first inlet port 120a′ and a second inlet port 120a″. It is also contemplated and within the scope of the present disclosure for the proximal end of the inlet tubing 120 to be a single tube with dual lumen arranged either concentrically or eccentrically. Flow restrictor 135 is associated with the first inlet port 120a′, while a one-way valve 130 is associated with the second inlet port 120a″. Both inlet ports 120a, 120a″ empty into a collection vessel 140, preferably a disposable bag, for collecting fluid (e.g., saline during prepping/flushing prior to use and/or blood during use). Prior to use, the cyclic aspiration system may be prepped or flushed to eliminate air from the system by aspirating via the syringe 105 a relatively small amount of saline (or blood) from the system, as shown in FIG. 1B. To ensure that the system is free of air, the distal end of the inlet tubing 120 connected to the proximal end of the aspiration catheter is dipped into a dish of saline and the plunger 115 is retracted sucking or drawing the saline or blood into the syringe 105. Then the plunger 115 is advanced within the syringe 105 pushing saline or blood along the inlet tubing 120 towards the collection vessel 140. To maximize the overall volume capacity of the collection vessel 140 during aspiration, preferably during prepping or flushing the collection vessel is filled with no more than 5% saline.

Generation of the vacuum pressure interval of the cyclic aspiration pressure waveform is shown in FIG. 1C with the retraction or drawing back of the plunger or piston 115 within the barrel of the syringe 105 (as indicated by the directional arrow) producing the vacuum pressure in the syringe 105. Vacuum pressure is drawn, albeit unequally, between the aspiration catheter 100 and the collection vessel 140. That is, constriction of the inlet tubing 120 draws via the first inlet port 120a′ a minimal amount (e.g., ≤approximately 5%) of fluid from the collection vessel 140 (as indicated by the thin directional arrows pointing in the distal direction) resulting in negligible, de minimis, or insignificant loss or reduction of suction from the distal tip of the aspiration catheter 100. The remaining suction (e.g., ≥approximately 95%) (as indicated by the thick directional arrows pointing in the proximal direction) is pulled from the distal tip/end of the aspiration catheter 100 drawing the clot 165 thereto.

Generation of the positive pressure interval of the cyclic aspiration pressure waveform is illustrated in FIG. 1D with the clot 165 lodged at the distal tip/end of the aspiration catheter 100. Positive pressure is generated by the advancement of the plunger or piston 115 (as indicated by the directional arrow) in the barrel of the syringe 105 forcing the positive pressure, albeit unequally, into the aspiration catheter 100 and the collection vessel 140. Specifically, a minimal amount (e.g., ≤approximately 5%) (as indicated by the thin directional arrow pointing in the proximal direction) of the forced positive pressure enters the collection vessel 140 via the first inlet port 120a′ resulting in negligible, de minimis, or insignificant loss or reduction of forced positive pressure applied to distal tip of the aspiration catheter. The remaining forced positive pressure (e.g., ≥approximately 95%) (as indicated by the thick directional arrows pointing in the distal direction) is applied to the distal tip/end of the aspiration catheter 100 expelling the clot 165. When the positive pressure in the second inlet port 120a″ exceeds the predetermined set pressure (e.g., 10000 pa) of the one-way 130 (i.e., unidirectional) valve opens. Selecting a one-way valve having a relative low predetermined set pressure allows the aspirated blood in the system to enter the collection vessel 140 at relatively low pressures when a clot is not lodged at the distal tip/end of the aspiration catheter. Even though the one-way valve 130 opens at the predetermined set pressure (e.g., 10000 pa), the positive pressure in the cyclic aspiration system may reach significantly higher pressures exceeding the fundamental flow rate limit through the one-way valve 130 (e.g., when the clot is lodged at the distal tip/end of the aspiration catheter, as depicted in FIG. 1D). If the syringe 105 is applying a flow rate exceeding the fundamental flow rate that the one-way valve is able to dissipate, then the positive pressure in the cyclic aspiration system will build up or increase in the second inlet port 120a″ (as indicated by the thicker directional arrow pointing in the proximal direction FIG. 1D).

FIG. 1E shows a reciprocating plunger or piston 115 cyclically retracted or advanced by a spring-loaded rotating wheel 150 (i.e., linear displacement mechanism). Different size or volume collection vessels 140 (e.g., disposable collection bags (e.g., low, medium, and high) may be sold separately or together for collecting low, medium, and high volumes of aspirated fluid (e.g., blood and/or saline). In addition, multiple systems like that shown in FIG. 1A may be connected together in series to accommodate larger volumes of aspirated fluid. This simplistic system advantageously employs only syringes which physicians and interventionalist have a known familiarity with using. Preferably, the overall cost of manufacture of this simplistic system is inexpensive that the components (e.g., collection vessel, syringe, plunger or piston, inlet tubing, connector, catheter hub and aspiration catheter may be either separately or as an assembled unit/module discarded or disposed of after a single use. A relatively large capacity syringe, e.g., 60 ml, is preferably used without having to be replaced. One or more non-disposable, reusable electronic components, such as a pressure sensor, variable restrictor, wireless dongle, batteries, and a motor, may be used with the system without becoming contaminated by blood separate from the module of disposable components contaminated by blood. If the motor is not employed, those other non-disposable electronic components in the cyclic aspiration system operate using less power or energy. The example in FIG. 1E depicts the cyclic aspiration system with the aspirator catheter 100 connected to a rotating hemostatic valve 103 (instead of a proximal hub 103 shown in FIGS. 1A-1D). Also, a clot capture device (e.g., a stentriever) may be positioned within the inlet tubing 120 to assist with removal of the aspirated clot therein.

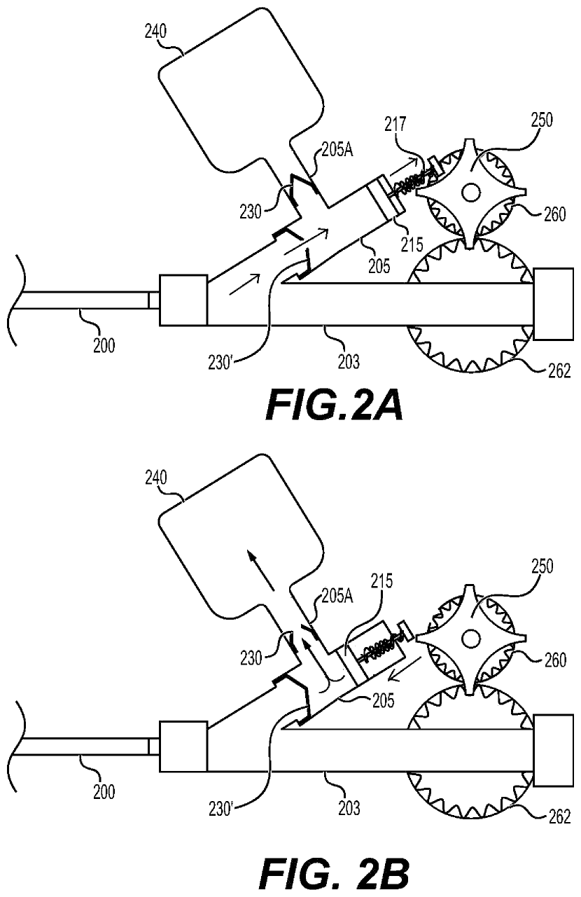

Instead of the piston or plunger displaceable within the barrel of the syringe associated with inlet tubing, in an alternative cyclic aspiration system in accordance with the present disclosure the plunger or piston may be displaceable within a port of a rotating hemostatic valve (RHV), as shown in FIGS. 2A & 2B. A reciprocating plunger or piston 215, shown in a retracted state, is displaceable within a pressure port 205 of a rotating hemostatic valve which may optionally include a main port 203 for receiving therethrough an auxiliary device (e.g., stentriever). Plunger or piston 215 is preferably spring-loaded 217 to restore to a natural or default retracted state when not subject to an external force. The plunger 215 is reciprocatingly actuated (i.e., advanced in the pressure port 205 of the RHV) when intermittently engaging with an actuating wheel 250 (i.e., linear displacement mechanism). In FIG. 2A, rotation of the actuating wheel 250 is accomplished using an arrangement of spur gears manually powered. Specifically, the actuating wheel 250 is mounted to a first gear (i.e., pinion) 260 driven by a second gear (i.e., wheel) 262 manually powered via a round spiral spring. Activation or release of the spiral spring powers the wheel 262 which, in turn, drives the pinion 260 thereby rotating the actuating wheel 250 mounted thereto. The spiral spring allows for a predetermined amount of time, e.g., approximately 2 minutes, the continuous running of the cyclic aspiration system at a constant rate. In operation, during continuous rotation, the actuating wheel 250 intermittently cyclically disengages and engages with the plunger 215. During disengagement (i.e., free of application of an external force by the actuation wheel 250) the spring-loaded plunger 215 is automatically restored to the natural or default retracted state generating the vacuum pressure in the RHV. Whereas, during engagement (i.e., imposing an external force) the plunger 215 is advanced in a distal direction in the pressure port 205 of the RHV compressing the aspirated fluid collected therein generating the positive pressure. While undergoing vacuum pressure, the aspirated fluid in the system may be collected or stored in a collection vessel 240, preferably a disposable bag, connected to a side port 205a of the pressure port 205 of the RHV. Arranged within the side port 205a is a first backflow prevention device 230 (e.g., duckbill valve, one-way valve, non-return valve, check valve, reflux valve, retention valve, etc.) that opens under vacuum pressure allowing fluid to be drawn or sucked into the collection vessel 240. While under positive pressure the duckbill valve 230 prevents fluid to flow back into the aspiration catheter. A second backflow prevention device 230′ (e.g., duckbill valve, one-way valve, non-return valve, check valve, reflux valve, retention valve, etc.) is disposed in the pressure port 205. While the fluid is compressed by the plunger 215 being advanced in the pressure port 205, the fluid is prevented from back flow therethrough. Optionally, when closed it is possible for there to be a small hole or opening in the duckbill valve 230′ to permit passage therethrough of slightly more of the generated positive pressure while still preventing the back flow of fluid. Since the plunger or piston 215 is at the RHV attached to the aspiration catheter 200 there is minimum decay of the positive pressure prior to reaching the distal tip/end. Accordingly, a minimum volume of fluid need be displaced (i.e., compressed) by the plunger or piston 215 to achieve relatively high levels of positive pressure at the distal tip/end at the aspiration catheter.

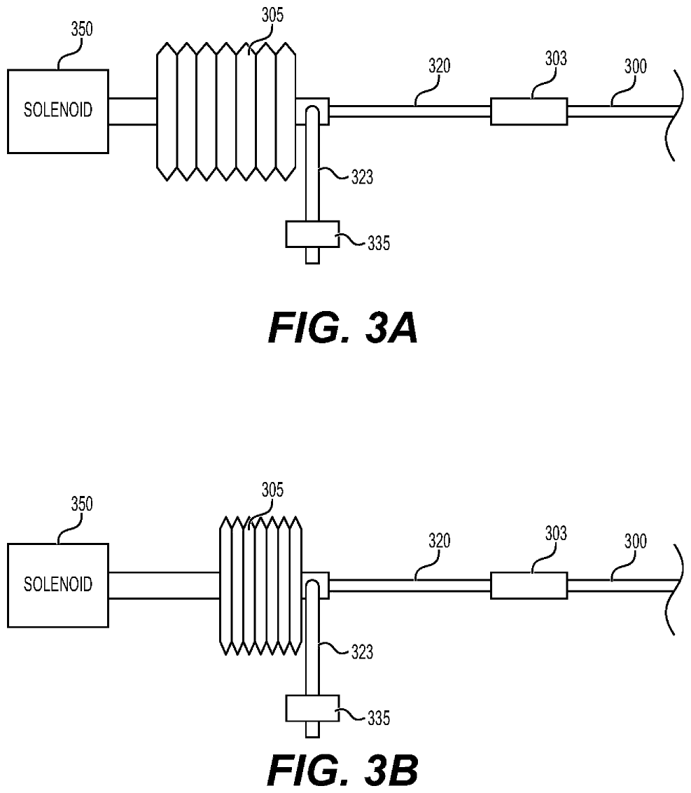

In still yet another example in FIGS. 3A & 3B of the cyclic aspiration system in accordance with the present disclosure, once again the need for a vacuum pump is eliminated. The cyclic aspiration pressure waveform is generated using a concertinaed (“flexi”) vessel connected in fluid communication to the inlet tubing disposed proximally of the catheter hub 303. In the example in FIG. 3A depicts the example concertinaed vessel 305 (e.g., a bellows, concertina, or accordion) axially collapsible when subject to an external axial force imposed by a linear displacement mechanism 350 (e.g., linear actuator, solenoid, cam, a rotation to reciprocating mechanism, reciprocating mechanism, etc.) externally arranged and secured thereto (FIG. 3B). The concertinaed vessel 305 preferably has an axial resistance so that when free (i.e., upon withdraw or removal) of the external axial compression force the concertinaed vessel 305 is automatically restored to a natural or default non-compressed state (FIG. 3A). During withdraw of the external axial compression force depicted in FIG. 3A, due to the axial resistance, the concertinaed vessel 305 is restored to its default non-compressed (i.e., axially expanded) state producing the vacuum pressure interval of the cyclic aspiration pressure waveform. In response to the solenoid 313 imposing the external axial force sufficient to overcome the axial resistance, the concertinaed vessel 305 axially collapses compressing the fluid collected therein creating the interval of positive pressure, as illustrated in FIG. 3B. A variable valve 335 allows depressurization of the system (i.e., evacuation of the positive pressure eventually returning to blood pressure) in preparation of reapplying or restoring the vacuum pressure during expansion of the concertinaed vessel 305 to its natural (i.e., non-collapsed) state. Furthermore, in the case where the linear displacement mechanism 350 (e.g., solenoid) employed applies a constant force per stroke, the variable valve 335 may be used to control or vary the amplitude of the generated positive pressure in the system.

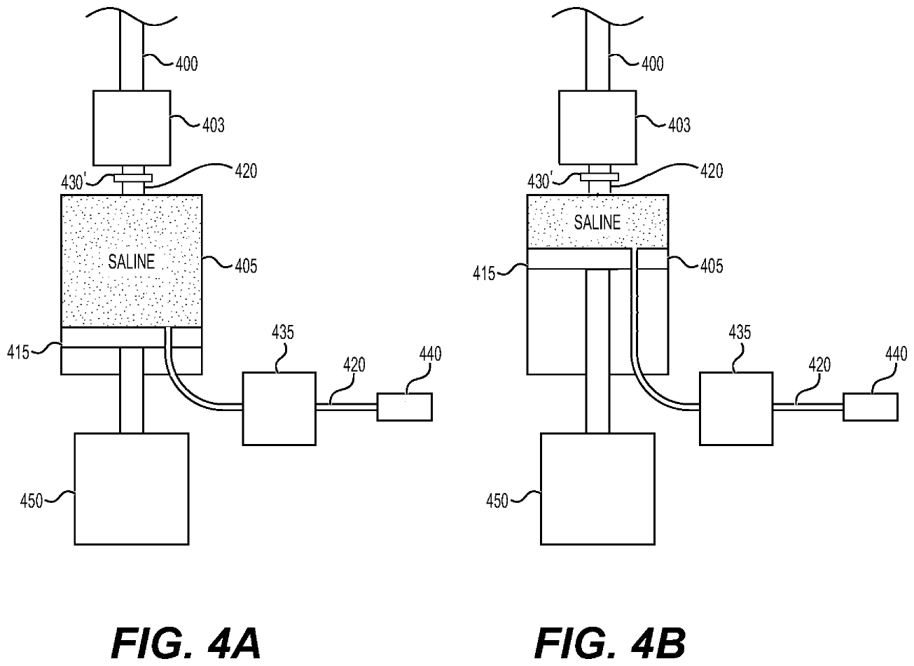

While still yet another example cyclic aspiration system in FIGS. 4A & 4B generates the cyclic aspiration pressure waveform using a plunger or piston 415 slidable within a reservoir 405 arranged proximally of the catheter hub 403 with a one-way valve 430 disposed therebetween. Retraction of the plunger 415 in the reservoir 405 generates vacuum pressure opening the one-way valve 430 allowing the fluid from the aspiration catheter 400 to be drawn or sucked therethrough. When the plunger 415 is advanced in the reservoir 405 the positive pressure pushing towards the aspiration catheter 400 closes the one-way valve 430 preventing fluid from being injected into the aspiration catheter and preventing the clot from being pushed too far forward, The plunger of piston 415 is intermittently cyclically displaceable (e.g., retracted or advanced) via a linear displacement mechanism 450 (e.g., linear actuator, solenoid, cam, a rotation to reciprocating mechanism, reciprocating mechanism, etc.) arranged externally of the reservoir 405. Prior to use during endovascular treatment to capture a clot (i.e., pre-treatment), the reservoir 405 may be prefilled or preloaded with saline for initial prepping or flushing (i.e., pre-treatment). In operation, the solenoid 450 intermittently cyclically: (i) retracts the plunger or piston 415 within the reservoir 405 creating vacuum pressure drawing fluid (e.g., blood and/or saline) into the reservoir 405; and (ii) advances the plunger or piston 415 within the reservoir 405 compressing the fluid collected therein generating the positive pressure. Repeated cyclic retraction and advancement of the plunger or piston 415 produces the cyclic aspiration pressure waveform of intermittent cyclic intervals of vacuum pressure and positive pressure using the dual pressure generator receptacle without the use of a vacuum pump. Preferably the fluid collected in the reservoir 405 may be siphoned via inlet tubing 420 to a separate collection tank 440 (e.g., disposable bag). A variable valve 435 allows depressurization of the system (i.e., evacuation of the positive pressure eventually returning to blood pressure) in preparation of reapplying or restoring the vacuum pressure during retraction of the plunger 415. Furthermore, in the case where the linear displacement mechanism (e.g., solenoid 450) applies a constant force per stroke, the variable valve 435 may be used to control or vary the amplitude of the generated positive pressure in the system. Variable valve 435 (e.g., one-way valve) serves a dual function: (i) acting as a one-way valve so that fluid is only sent to the tank 440, while preventing fluid from being sucked from the tank 440; and (ii) acting as a variable restrictor or pressure control valve controlling the pressure within the reservoir 405 and thus pressure delivered to the aspiration catheter 400.

Preferably, the two linear displacement mechanisms 450, 455 are synchronized with one another to allow a seamless transition between the positive pressure and the vacuum pressure. In operation, FIG. 4A depicts the plunger or piston 415 while in a retracted state within the reservoir 405 generating an interval of vacuum pressure. In response to the solenoid 450 applying an external axial force the plunger or piston 415 is advanced within the reservoir 405 compressing the fluid collected therein generating the interval of positive pressure. Advantageously, those components of the cyclic aspiration system contaminated by blood (e.g., inlet tubing 420, liquid reservoir 405, plunger 415, inlet/auxiliary tubing 420, and collection vessel 440) are inexpensive and discardable or disposable after a single use (preferably together as a single module or unit), whereas those re-usable, non-disposable electronic components linear displacement mechanism(s) 450, 455 (e.g., solenoid) are arranged external thereof and thus not contaminated (not physically contacted) by blood.

Aspects of the present disclosure are also provided by the following numbered clauses:

### Clause 1

A cyclic aspiration system for producing a cyclic aspiration pressure waveform of intermittent cycles of vacuum pressure below atmospheric pressure and positive pressure higher than vacuum pressure, the cyclic aspiration system comprising: an aspiration catheter (100, 200, 300, 400) having a proximal end and an opposite distal end; and a dual pressure generator receptacle (105, 205, 305, 405) disposed proximally of and connected in fluid communication with the aspiration catheter (100, 200, 300, 400); the dual pressure generator receptacle (105, 205, 305, 405) receiving a collectable fluid therein that is intermittently cyclically subjectable to application or withdraw of a compressible force via a linear displacement mechanism (150, 250, 350, 450); the positive pressure being generatable while the collectable fluid in the dual pressure generator receptacle (105, 205, 305, 405) is subject to the compressive force and the vacuum pressure being generatable while the collectable fluid in the dual pressure generator receptacle is not subject to the compressive force; wherein the cyclic aspiration pressure waveform is producible via the dual pressure generator receptacle without a separate vacuum pump.

### Clause 2

The cyclic aspiration system of Clause 1, further comprising inlet tubing (120, 420) disposed proximally of a proximal hub (103, 303, 403) arranged at the proximal end of the aspiration catheter (100, 400); and the dual pressure generator receptacle (105, 405) and a separate collection vessel (140, 440) is connected in fluid communication with the inlet tubing (120, 420).

### Clause 3

The cyclic aspiration system of Clause 2, wherein the dual pressure generator receptacle is a syringe (105) or a reservoir (405) having a plunger (115, 415) displaceable therein via the linear displaceable mechanism (150, 450); and the collection vessel (140, 440) is a tank or disposable bag in fluid communication therewith via the inlet tubing (120, 420).

### Clause 4

The cyclic aspiration system of any of Clauses 2 through 3 in accordance with claim 2, further comprising a first one-way valve (130′, 430′) disposed in the inlet tubing (120, 420) to prevent fluid collectable in the system from passing distally therethrough into the aspiration catheter (100, 400) when subject to the positive pressure.

### Clause 5

The cyclic aspiration system of Clause 4, further comprising a second one-way valve (130, 430) preventing fluid collectable in the collection vessel (140, 440) from exiting and controlling the positive pressure to the aspiration catheter (100, 400).

### Clause 6

The cyclic aspiration system of Clause 1, further comprising a rotating hemostatic valve having an outlet port and a pressure port (205) with a side port (205a); the proximal end of the aspiration catheter (200) is in fluid communication with the outlet port of the rotating hemostatic valve; wherein the dual pressure generator receptacle is the pressure port (205) of the rotating hemostatic valve; and wherein the linear displacement mechanism cyclically engages with a plunger (215) displaceable within the pressure port (205) of the rotating hemostatic valve.

### Clause 7

The cyclic aspiration system of Clause 6, further comprising a first one-way valve (230′) disposed in the inlet tubing to prevent fluid collectable in the system from passing distally therethrough into the aspiration catheter (200) when subject to the positive pressure.

### Clause 8

The cyclic aspiration system of Clause 7, further comprising a second one-way valve (230) preventing fluid collectable in the collection vessel (240) from exiting and controlling the positive pressure to the aspiration catheter (200).

### Clause 9

The cyclic aspiration system of Clause 1, wherein the dual pressure generator receptacle is a concertinaed vessel (305) having an axial resistance; the concertinaed vessel (305) is connected via inlet tubing (320) to a proximal hub (303) arranged at the proximal end of the aspiration catheter (300); and the linear displacement mechanism (350) imposes or withdraws the compressible force on the concertinaed vessel (305) transitionable between an axially collapsed state and an axially expanded state.

### Clause 10

The cyclic aspiration system of any of Clauses 1 through 9, wherein the linear displacement mechanism (150, 250, 350, 450) is external of the dual pressure generator receptacle (105, 205, 305, 405), not contaminatable with blood, and reusable; while the dual pressure generator receptacle is contaminatable with blood and discardable after a single use.

### Clause 11

A method for using a cyclic aspiration system for producing a cyclic aspiration pressure waveform of intermittent cycles of vacuum pressure below atmospheric pressure and positive pressure higher than vacuum pressure; the cyclic aspiration system comprising: an aspiration catheter (100, 200, 300, 400) having a proximal end and an opposite distal end; and a dual pressure generator receptacle (105, 205, 305, 405) disposed proximally of and connected in fluid communication with the aspiration catheter (100, 200, 300, 400); wherein the method comprises the steps of: delivery of the aspiration catheter (100, 200, 300, 400) through a vessel to a target site on a proximal side of a clot; and producing the cyclic aspiration pressure waveform using the dual pressure generator receptacle (105, 205, 305, 405) by intermittently cyclically subjecting a collectable fluid receivable therein to application or withdraw of a compressible force via a linear displacement mechanism (150, 250, 350, 450); the positive pressure being generated while the collectable fluid in the dual pressure generator receptacle (105, 205, 305, 405) is subject to the compressive force and the vacuum pressure being generated while the collectable fluid in the dual pressure generator receptacle (105, 205, 305, 405) is not subject to the compressive force; wherein the cyclic aspiration pressure waveform is producible via the dual pressure generator receptacle (105, 205, 305, 405) without a separate vacuum pump.

### Clause 12

The method of Clause 11, wherein the cyclic aspiration system further comprises inlet tubing (120, 420) disposed proximally of a proximal hub (103, 303, 403) arranged at the proximal end of the aspiration catheter (100, 400); and the dual pressure generator receptacle (105, 405) and a separate collection vessel (140, 440) is connected in fluid communication with the inlet tubing (120, 420).

### Clause 13

The method of Clause 12, wherein the dual pressure generator receptacle is a syringe (105) or a reservoir (405) having a plunger (115, 415) displaceable therein via the linear displaceable mechanism (150, 450); and the collection vessel (140, 440) is a tank or disposable bag in fluid communication therewith via the inlet tubing (120, 420).

### Clause 14

The method of any of Clauses 12 through 13, further comprising a first one-way valve (130′, 430′) disposed in the inlet tubing (120, 420) to prevent fluid collectable in the system from passing distally therethrough into the aspiration catheter (100, 400) when subject to the positive pressure.

### Clause 15

The method of Clause 14, further comprising a second one-way valve (130, 430) preventing fluid collectable in the collection vessel (140, 440) from exiting and controlling the positive pressure to the aspiration catheter (100, 400).

### Clause 16

The method of Clause 11, wherein the cyclic aspiration system further comprises a rotating hemostatic valve having an outlet port and a pressure port (205) with a side port (205a); the proximal end of the aspiration catheter (200) is in fluid communication with the outlet port of the rotating hemostatic valve; wherein the dual pressure generator receptacle is the pressure port (205) of the rotating hemostatic valve; and wherein the linear displacement mechanism cyclically engages with a plunger (215) displaceable within the pressure port (205) of the rotating hemostatic valve.

### Clause 17

The method of Clause 16, further comprising a first one-way valve (230′) disposed in the inlet tubing to prevent fluid collectable in the system from passing distally therethrough into the aspiration catheter (200) when subject to the positive pressure.

### Clause 18

The method of Clause 17, further comprising a second one-way valve (230) preventing fluid collectable in the collection vessel (240) from exiting and controlling the positive pressure to the aspiration catheter (200).

### Clause 19

The method of Clause 11, wherein the dual pressure generator receptacle is a concertinaed vessel (305) having an axial resistance; the concertinaed vessel (305) is connected via inlet tubing (320) to a proximal hub (303) attached to the aspiration catheter (300); and the linear displacement mechanism (350) imposes or withdraws the compressible force on the concertinaed vessel (305) transitionable between an axially collapsed state and an axially expanded state.

### Clause 20

The method of any of clauses 11 through 19, wherein the linear displacement mechanism (150, 250, 350, 450) is external of the dual pressure generator receptacle (105, 205, 305, 405), not contaminatable with blood, and reusable; while the dual pressure generator receptacle is contaminatable with blood and discardable after a single use.

The descriptions contained herein are examples and are not intended in any way to limit the scope of the present disclosure. As described herein, the present disclosure contemplates many variations and modifications of a cyclic aspiration system for generating a cyclic aspiration pressure waveform without using a vacuum pump (e.g., centrifugal pump, piston pump, or diaphragm pump). In particular, the cyclic aspiration system generates the cyclic aspiration pressure waveform using a dual pressure generator receptacle (e.g., syringe in fluid communication with inlet tubing, a displaceable plunger disposed with the pressure port of an RHV, concertinaed vessel, or reservoir with displaceable plunger slidable therein). The cyclic aspiration pressure waveform of intermittent cyclic intervals of vacuum pressure (i.e., below atmospheric pressure) and positive pressure (i.e., higher than vacuum pressure) being generated by intermittently cyclically withdrawing and applying compressive force to the fluid collected in the dual pressure generator receptacle. Modifications and variations apparent to those having skilled in the pertinent art according to the teachings of this disclosure are intended to be within the scope of the claims which follow.

What is claimed is:

1. A cyclic aspiration system for producing a cyclic aspiration pressure waveform of intermittent cycles of vacuum pressure below atmospheric pressure and positive pressure higher than vacuum pressure, the cyclic aspiration system comprising:
an aspiration catheter having a proximal end and an opposite distal end; and
a dual pressure generator receptacle disposed proximally of and connected in fluid communication with the aspiration catheter; the dual pressure generator receptacle receiving a collectable fluid therein that is intermittently cyclically subjectable to application or withdraw of a compressible force via a linear displacement mechanism; the positive pressure being generatable while the collectable fluid in the dual pressure generator receptacle is subject to the compressive force and the vacuum pressure being generatable while the collectable fluid in the dual pressure generator receptacle is not subject to the compressive force;
wherein the cyclic aspiration pressure waveform is producible via the dual pressure generator receptacle without a separate vacuum pump.

2. The cyclic aspiration system in accordance with claim 1, further comprising inlet tubing disposed proximally of a proximal hub arranged at the proximal end of the aspiration catheter; and the dual pressure generator receptacle and a separate collection vessel is connected in fluid communication with the inlet tubing.

3. The cyclic aspiration system in accordance with claim 2, wherein the dual pressure generator receptacle is a syringe or a reservoir having a plunger displaceable therein via the linear displaceable mechanism; and the collection vessel is a tank or disposable bag in fluid communication therewith via the inlet tubing.

4. The cyclic aspiration system in accordance with claim 2, further comprising a first one-way valve disposed in the inlet tubing to prevent fluid collectable in the system from passing distally therethrough into the aspiration catheter when subject to the positive pressure.

5. The cyclic aspiration system in accordance with claim 4, further comprising a second one-way valve preventing fluid collectable in the collection vessel from exiting and controlling the positive pressure to the aspiration catheter.

6. The cyclic aspiration system in accordance with claim 1, further comprising a rotating hemostatic valve having an outlet port and a pressure port with a side port; the proximal end of the aspiration catheter is in fluid communication with the outlet port of the rotating hemostatic valve; wherein the dual pressure generator receptacle is the pressure port of the rotating hemostatic valve; and wherein the linear displacement mechanism cyclically engages with a plunger displaceable within the pressure port of the rotating hemostatic valve.

7. The cyclic aspiration system in accordance with claim 6, further comprising a first one-way valve disposed in the inlet tubing to prevent fluid collectable in the system from passing distally therethrough into the aspiration catheter when subject to the positive pressure.

8. The cyclic aspiration system in accordance with claim 7, further comprising a second one-way valve preventing fluid collectable in the collection vessel from exiting and controlling the positive pressure to the aspiration catheter.

9. The cyclic aspiration system in accordance with claim 1, wherein the dual pressure generator receptacle is a concertinaed vessel having an axial resistance; the concertinaed vessel is connected via inlet tubing to a proximal hub arranged at the proximal end of the aspiration catheter; and the linear displacement mechanism imposes or withdraws the compressible force on the concertinaed vessel transitionable between an axially collapsed state and an axially expanded state.

10. The cyclic aspiration system in accordance with claim 1, wherein the linear displacement mechanism is external of the dual pressure generator receptacle, not contaminatable with blood, and reusable; while the dual pressure generator receptacle is contaminatable with blood and discardable after a single use.

11. A method for using a cyclic aspiration system for producing a cyclic aspiration pressure waveform of intermittent cycles of vacuum pressure below atmospheric pressure and positive pressure higher than vacuum pressure; the cyclic aspiration system comprising: an aspiration catheter having a proximal end and an opposite distal end; and a dual pressure generator receptacle disposed proximally of and connected in fluid communication with the aspiration catheter; wherein the method comprises the steps of:
delivery of the aspiration catheter through a vessel to a target site on a proximal side of a clot; and
producing the cyclic aspiration pressure waveform using the dual pressure generator receptacle by intermittently cyclically subjecting a collectable fluid receivable therein to application or withdraw of a compressible force via a linear displacement mechanism; the positive pressure being generated while the collectable fluid in the dual pressure generator receptacle is subject to the compressive force and the vacuum pressure being generated while the collectable fluid in the dual pressure generator receptacle is not subject to the compressive force; wherein the cyclic aspiration pressure waveform is producible via the dual pressure generator receptacle without a separate vacuum pump.

12. The method in accordance with claim 11, wherein the cyclic aspiration system further comprises inlet tubing disposed proximally of a proximal hub arranged at the proximal end of the aspiration catheter; and the dual pressure generator receptacle and a separate collection vessel is connected in fluid communication with the inlet tubing.

13. The method in accordance with claim 12, wherein the dual pressure generator receptacle is a syringe or a reservoir having a plunger displaceable therein via the linear displaceable mechanism; and the collection vessel is a tank or disposable bag in fluid communication therewith via the inlet tubing.

14. The method in accordance with claim 12, further comprising a first one-way valve disposed in the inlet tubing to prevent fluid collectable in the system from passing distally therethrough into the aspiration catheter when subject to the positive pressure.

15. The method in accordance with claim 14, further comprising a second one-way valve preventing fluid collectable in the collection vessel from exiting and controlling the positive pressure to the aspiration catheter.

16. The method in accordance with claim 11, wherein the cyclic aspiration system further comprises a rotating hemostatic valve having an outlet port and a pressure port with a side port; the proximal end of the aspiration catheter is in fluid communication with the outlet port of the rotating hemostatic valve; wherein the dual pressure generator receptacle is the pressure port of the rotating hemostatic valve; and wherein the linear displacement mechanism cyclically engages with a plunger displaceable within the pressure port of the rotating hemostatic valve.

17. The method in accordance with claim 16, further comprising a first one-way valve disposed in the inlet tubing to prevent fluid collectable in the system from passing distally therethrough into the aspiration catheter when subject to the positive pressure.

18. The method in accordance with claim 17, further comprising a second one-way valve preventing fluid collectable in the collection vessel from exiting and controlling the positive pressure to the aspiration catheter.

19. The method in accordance with claim 11, wherein the dual pressure generator receptacle is a concertinaed vessel having an axial resistance; the concertinaed vessel is connected via inlet tubing to a proximal hub attached to the aspiration catheter; and the linear displacement mechanism imposes or withdraws the compressible force on the concertinaed vessel transitionable between an axially collapsed state and an axially expanded state.

20. The method in accordance with claim 11, wherein the linear displacement mechanism is external of the dual pressure generator receptacle, not contaminatable with blood, and reusable; while the dual pressure generator receptacle is contaminatable with blood and discardable after a single use.
